(19) United States
(12) Patent Application Publication
(10) Pub. No.: US 2012/0213720 A1
(43) Pub. Date: Aug. 23, 2012

(54) Prodrug composition for skin with twin protecting groups bound by disubstituted benzene

(76) Inventor: Hungsen Lai, Andover, MA (US)

(21) Appl. No.: 12/929,886

(22) Filed: Feb. 23, 2011

(51) Int. Cl. A61K 8/49; A61Q 19/08; A61Q 1/10; A61Q 1/14; C07D 405/12; A61Q 5/12

(52) U.S. Cl. 424/63; 549/415; 514/460; 424/70.1

(57) ABSTRACT

A prodrug composition for skin with twin protecting groups bound by disubstituted benzene is disclosed. The prodrug composition is a derivative of 1,4-bis(tetrahydro-2H-pyran-2-yloxy)benzene. Thereby, the twin protecting groups endow the prodrug composition with improved duration of action and efficiency to a targeted object such as melanocytes or tyrosinase, so as to facilitate distributing the prodrug composition over the stratum basale, in turn promoting melanocytes' absorption and tyrosinase's transportation as well as metabolism.

### BACKGROUND OF THE INVENTION

1. Technical Field

The present invention relates to a prodrug composition for skin with twin protecting groups bound by disubstituted benzene, and more particularly, the prodrug composition acts as a prodrug of deoxy-arbutin. The so-called prodrug is a compound, when not in vivo, being inactive or less active as compared with its parent drug (i.e. disubstituted benzene, such as hydroquinone). The compound and the parent drug (disubstituted benzene) are connected mutually with a covalent bond to form a new derivative composition (i.e. the prodrug composition for skin disclosed in the present invention). The derivative composition in vivo is metabolized by enzyme, and releases the active substance (i.e. deoxy-arbutin) of the parent drug so as to give its pharmacological effect by suppressing the activity of tyrosinase of melanocytes in the skin, thereby regulating the inhibition of melanin synthesis.

2. Description of Related Art

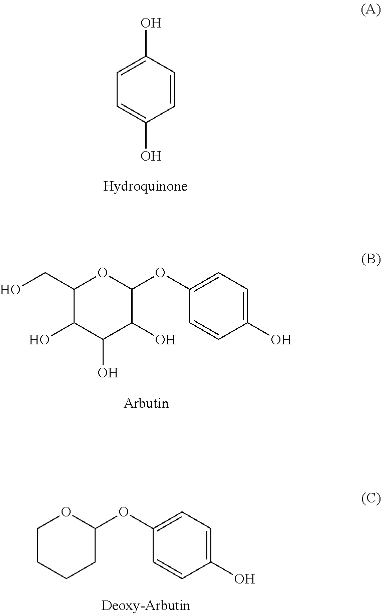

Traditionally, the whitening cosmetics used in hospitals and beauty salons use hydroquinone (hereinafter referred to as HQ, as represented by the above formula (A)) as a tyrosinase inhibitor that inhibits melanin synthesis. While HQ suppresses the activity of tyrosinase and inhibits melanin synthesis by destroying melanocytes, it has strong toxicity and is highly irritative to the skin. Recently, in view that its long-term and high-concentration use tends to bring about side effects such as dermatitis, some countries have forbidden its use in whitening cosmetics.

Thus, a deoxy-arbutin compound (hereinafter referred to as DA, as represented by the above formula (C)) has been developed (referring to Reference 1 through Reference 3 as recited below). Such a compound is derived from arbutin (as represented by the above formula (B)), which is extracted from leaves of Arbutus. DA has a safety concentration four times higher than that of HQ, meaning that, as compared with HQ, DA is less irritative to the skin and less toxic to melanocytes in the skin. In addition, after stop of usage of DA, tyrosinase in the skin can gradually restore to recovery the melanin content. This fact indicates that DA does not kill melanocytes and leaves melanocytes with their normal physiological mechanisms. A clinical research for 8 weeks has shown that local usage of DA gave a progressive and apparent effect of whitening the skin, and that DA solutions with proper concentration presented desired clinical effects in three weeks. Also, according to experiments, DA has an anti-melanin effect 10 times higher than that of HQ and 350 times higher than that of normal arbutin. Thus, DA is ideal for replacing HQ as a safer preparation material.

DA is generated by deoxidizing the glycoside of arbutin. Thus, while there are differences between DA, arbutin and HQ in formula, the three suppress the activity of tyrosinase in the same manner. However, DA is relatively instable and it releases toxic HQ. In addition, when catalyzed by external factors such as alkaline solutions, trace metal ions and free radicals in the air, DA is likely to get oxidized and darkened (browned or blacked). As such, DA is unfavorable for long-term storage, and can significantly degrade the appearance of DA-containing cosmetic products. As a remedial measure, DA has to be stabilized by, for example, addition of an anti-oxidant before permeating into the stratum basale to be absorbed by melanocytes. Furthermore, as disclosed in Reference 4, DA has a half-life of 22.24 days at the room temperature of 25° C., and a reduced half-life of 7.11 days at 45° C., meaning that DA, when existing in an elevated temperature higher than the room temperature, tends to be oxidized and become instable. From this respect, DA has inferior chemical stability and is prone to be darkened when meeting oxygen and heat. Although anti-oxidants may somehow be a helpful technical means for restraining DA from darkening cosmetic products, the darkening problem of DA itself remains unsolved.

In view of this, the inventor of the present invention has done researches so as to herein propose a prodrug composition for skin with twin protecting groups bound by disubstituted benzene in order to overcome the shortcomings of existing deoxy-arbutin (DA), such as less preservable and giving hydroquinone (HQ), while being useful in reducing foxiness and freckles from the skin.

Reference 1: S. H. Hamed, P. Sriwiiyanont, R. R. Wickett, and R. Boissy, “Effect of DeoxyArbutin on Melanogenesis: In Vivo Comparision with other Melanogenesis Inhibitor”, Journal of Cosmetic Science, 2003 Annual Scientific Meeting, 118-119 (2003).

Reference 2: R. E. Boissy, M. Visscher, and M. A. deLong, “DeoxyArbutin: a novel reversible tyrosinase inhibitor with effective in vivo skin lightening potency”, Experimental Dermatology 2005: 14: 601-608 (2005).

Reference 3: S. H. Hamed, P. Sriwiiyanont, M. A. deLong, M. O. Visscher, R. R. Wickett, and R. E. Boissy, “Comparative efficacy and safety of deoxyarbutin, a new tyrosinase-inhibiting agent”, J. Cosmet. Sci., 57, 291-308 (July/August 2006).

Reference 4: Chao-Hsun Yang, Yi-Shyan Chen, Jeng-Shiow Lai, Willy W. L. Hong and Chih-Chien Lin, “Determination of the Thermodegradation of deoxyArbutin in Aqueous Solution by High Performance Liquid Chromatography”, Int. J. Mol. Sci. 2010, 11, 3977-3987.

### SUMMARY OF THE INVENTION

The primary objective of the present invention is to provide a prodrug composition for skin with twin protecting groups bound by disubstituted benzene. The prodrug composition is lipophilic and absorbable to the skin, while having good selectivity, high safety as well as outstanding pharmacodynamic properties that prolongs the duration of action. The prodrug composition after absorbed by the skin is decomposed into deoxy-arbutin that goes down to the stratum basale for inhibiting melanin synthesis, thereby remedying the shortcomings of existing deoxy-arbutin (DA), such as less preservable and giving hydroquinone (HQ), and being useful in reducing foxiness and freckles from the skin.

To this end, the prodrug composition for skin with twin protecting groups bound by disubstituted benzene comprises a composition represented by the following formula (I),

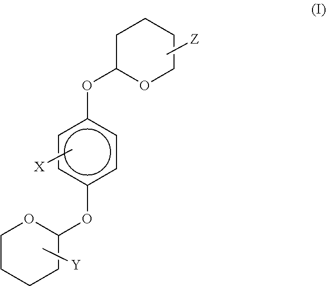

where, X is an independent benzene ring compound selected form disubstituted benzene or benzoic acid derivatives; Y is an independent heterocyclic compound with three- to six-membered rings and selected from an oxygen containing heterocyclic compound and a sulphur containing heterocyclic compound; and Z is an independent heterocyclic compound with three- to six-membered rings and selected from an oxygen containing heterocyclic compound and a sulphur containing heterocyclic compound; and Z is an independent heterocyclic compound, wherein Z and Y are identical or different heterocyclic compounds.

Thereby, in virtue of the substance of formula (I), the prodrug composition of the present invention features not only for being as good at inhibiting melanin synthesis as the exiting DA, but also for being safe and less stimulative to the skin yet well reaching stratum basale and having prolonged action. The prodrug composition is an oily substance having good stability and applicable to various preparations. Hence, a cosmetic product with the substance of formula (I) is able to give an excellent yet safe whitening effect.

### BRIEF DESCRIPTION OF THE DRAWINGS

The invention as well as a preferred mode of use, further objectives and advantages thereof will be best understood by reference to the following detailed description of illustrative embodiments when read in conjunction with the accompanying drawings, wherein:

FIG. 1 is a graph of data about a first embodiment of the present invention analyzed by a high performance liquid chromatography;

FIG. 1A is a graph of data about a second embodiment of the present invention analyzed by a high performance liquid chromatography;

FIG. 2 through FIG. 7 are data graphs made from clinical tests performed with the prodrug composition of the present invention; and

FIG. 8 through FIG. 13 are photographs showing whitening effects exhibited on subjects.

### DETAILED DESCRIPTION OF THE INVENTION

The present invention proposes a prodrug composition for skin with twin protecting groups bound by disubstituted benzene comprises a composition represented by the following formula (I),

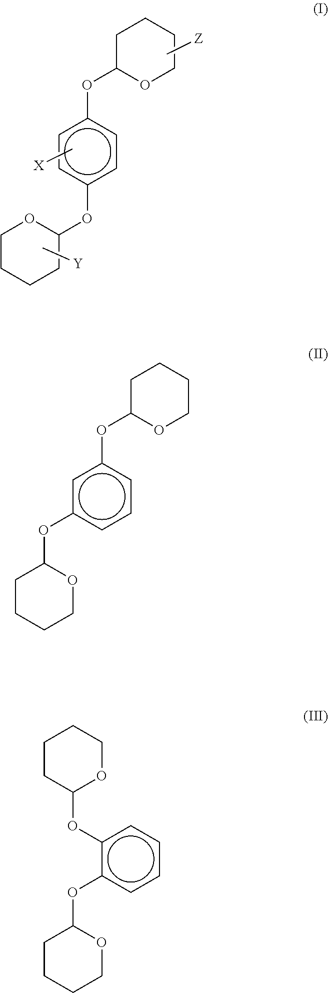

where, X is an independent benzene ring compound selected form disubstituted benzene or benzoic acid derivatives; Y is an independent heterocyclic compound with three- to six-membered rings and selected from an oxygen containing heterocyclic compound and a sulphur containing heterocyclic compound; and Z is an independent heterocyclic compound with three- to six-membered rings and selected from an oxygen containing heterocyclic compound and a sulphur containing heterocyclic compound; and Z is an independent heterocyclic compound. In some embodiments, Z and Y are identical or different heterocyclic compounds. The disclosed formula (I) has two isomerisms useful to cosmetic products, namely formula (II) and formula (III). In some embodiments, the disubstituted benzene is realized by a para-phenylene structure, a meta-phenylene structure or an ortho-phenylene structure, while, among the three formulas, the para-phenylene structure of the formula (I) is most preferable and the meta-phenylene structure of the formula (II) is also preferred. Thus, the following illustration is made by taking the formula (I) as an example. In the formula (I), the protecting groups are the heterocyclic compounds represented by Y and Z, and are both preferably tetrahydropyran (hereinafter referred to as THP). For the convenient of further illustration, the composition of formula (I) is herein called deoxy-arbutin plus, or DAP, as an abbreviation.

For the purpose of the present invention, the oxygen (O) containing heterocyclic compound is, for example, tetrahydropyran with a six-membered ring or tetrahydrofuran with a five-membered ring, while the sulphur (S) containing heterocyclic compound is, for example, tetrahydrothiopyran with a six-membered ring. The heterocyclic compound may have an alicyclic ring, or may have an aromatic ring, depending on practical needs, for changing the biological activity of the disclosed prodrug composition.

One simple method for making the prodrug composition of the present invention involves using deoxy-arbutin and dihydropyran provided by Girindus America Inc., Cincinnati, Ohio, USA to synthesize the composition of the present invention, in which process hydrogen ions are released from the hydroxyl groups (OH) of deoxy-arbutin, and coupled oxyl groups bind a carbon atom of dihydropyran, so as to form 1,4-bis(tetrahydro-2H-pyran-2-yloxy)benzene, namely the composition of formula (I) of the present invention, for giving outstanding water resistance and heat resistance.

Another scheme for preparing the disclosed DAP involves releasing hydrogen ions from two hydroxyl groups (OH) of hydroquinone (HQ) at the para positions to form a benzene ring with free oxyl groups, and releasing hydrogen ions from tetrahydropyran to be bound with the oxyl groups of the benzene ring, so as to produce the composition of the disclosed DAP with the protection provided by twin protecting groups.

In the synthesis, after releasing the hydrogen ions, the side-chain functional group (i.e. the hydroxyl group) of the benzene ring in the disubstituted benzene temporarily binds with the protecting groups to get protection, or when the entire hydroxyl group separates from the ring-like side chin of disubstituted benzene, other protecting groups are incorporated to replace the original hydroxyl group so as to form permanent protecting groups for providing protection.

In the formula (I) (i.e. Deoxy-Arbutin Plus, DAP) of the present invention, Z may have an independent chain compound replacing the heterocyclic compound, thus forming the formula (IV) of the present invention,

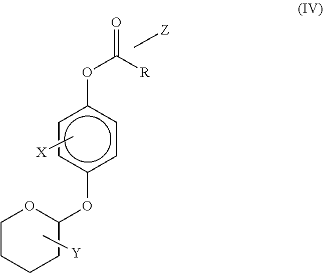

where the chain compound may be selected from acetyl (CH₃—C═O— or Ac—, composed of methyl and carbonyl groups), methyl, methoxy and methyl ether derivatives.

In the formula (IV), R may be selected form H, F, Cl, Br, NO₂, alkyl, aryl, aralkyl, alkoxy, aryloxy and disubstituted amine.

The disclosed prodrug composition has disubstituted benzene (such as hydroquinone) binding the twin protecting groups (two tetrahydropyran rings), so as to close organic functional groups and thereby prevent premature decomposition prior to in vivo enzyme reaction. Although this is not contributive to enhanced activity, the prodrug composition benefits from the changed physical and chemical properties that lead to elimination of unpleasing odor, reduction of the side effects related to the toxicity of the parent drug, and enhancement of selectivity as well as bioavailability acting on the targeted sites, which promotes the interaction between the prodrug composition and receptors or enzymes, thereby prolonging the duration of action and allowing the prodrug composition to be distributed over the stratum basale. Thus, melanocytes can well absorb the prodrug composition so as to improve tyrosinase's transportation and metabolism. In addition, the covalent bond binding the twin protecting groups with disubstituted benzene will undergo pyrolysis and be deprotected due to in-vivo enzyme reaction or enzyme catalysis, thus restoring the formerly protected disubstituted benzene to give the desired inhibition.

### First Embodiment

A first embodiment of the present invention is para-deoxy-arbutin plus of formula (I), with a synthetic route shown below,

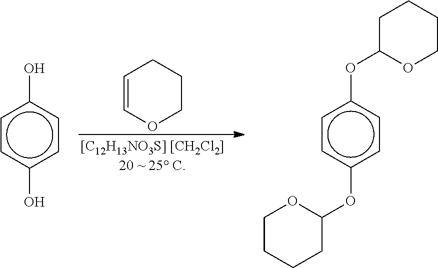

Along the synthetic route, hydroquinone (HQ) and 3,4-Dihydro-2H-Pyran were used as main materials, and pyridinium 4-toluenesulfonate was sued as the catalyst, while dichloromethane was used as the reactive solvent. The synthetic scale of the first embodiment was of 30 mole with conditions of 24 hours and the room temperature. The product of the above synthesis was then washed with 10% sodium hydroxide solution before crystallized and purified by dichloromethane and methanol.

The preparation steps and components involved by the first embodiment are given in detailed below:

### Step 1 of First Embodiment

3.30 kg of solid hydroquinone was weighted and dispersed in 20 L of liquid dichloromethane. Then at 20° C.˜25° C., liquid 3,4-Dihydro-2H-Pyran was added at an amount of 7.57 kg and solid pyridinium 4-toluenesulfonate was added at an amount of 45.2 g. Afterward, the mixture was stirred for reaction at 20° C.˜25° C. for 24 hours [Thin Layer Chromatography (TLC); Iodine Fuming Method]. In the first step, the iodine fuming method was implemented by sublimating iodine at the room temperature into purple gaseous molecules that are adsorbed by an object to be colored and thereby manifest the object.

### Step 2 of First Embodiment

After hydroquinone was removed (when TLC data showed that hydroquinone had Rf=0.42 and the disclosed Deoxy-Arbutin Plus had Rf=0.92), wash was performed with 10% sodium hydroxide solution in the organic phase for six times (each time with 5.0 L of the solution). Then wash was performed with water in the organic phase for three times (each time with 10 L of water, leading to an aqueous-phase product of about PH=7). After washed for three times with saturated NaCl solution (each time with 5.0 L of the saturated NaCl solution), the organic phase was added with 1.0 kg anhydrous magnesium sulfate and stirred for 12 hours to dry. For the instant step, said Rf is one of the chromatographic parameters for indicating component displacement, and defined by the ratio between solute migration distance and mobile phase migration distance. In TLC herein, Rf is the ratio between distances from an origin to a spot center and to the solvent front. Under particular chromatographic conditions, the Rf value of a specific compound is a constant and is thus a useful basis for identifying the compound.

### Step 3 of First Embodiment

The first filtration was performed and the cake was washed with dichloromethane for three times, each time with 1.0 L liquid dichloromethane. After concentrated under reduced pressure to produce dichloromethane fraction of 21.5 L, the organic phase at 20° C.˜25° C. was added with 5.0 L of methanol for beating. Then the second filtration was performed and the cake was washed with methanol for three times, each time with 2.0 L of methanol, so as to obtain the first product as white powder, with a wet weight of 8.5 kg. The mother solution was placed still in a cold bath of −10° C. for 30 minutes for crystallization before the third filtration. The cake was washed with methanol for three times, each time with methanol 1.0 L, so as to obtain the second product as white powder, with a wet weight of 5.2 kg. The mother solution (about 10 L) was concentrated under reduced pressure to produce fraction of 5.0 L, and then added with 2.0 L of methanol for beating before placed still in a cold bath of −10° C. for 60 minutes. Afterward, the fourth filtration was performed and the cake was washed with methanol for two times, each time with methanol 0.5 L, so as to obtain the third product as white powder, with a wet weight of 0.8 kg.

### Step 4 of First Embodiment

The three products as white powder of 14.5 kg were dried in vacuum to have a constant weight of 8.06 kg, with a yield of 97%, being the prodrug composition of the present invention, namely 1,4-bis(tetrahydro-2H-pyran-2-yloxy)benzene.

TABLE 1
List of Components of First Embodiment

| Component | | |
|---|---|---|
| (IUPAC Name/Chemical Formula) | Amount | |
| Hydroquinone (C₆H₄(OH)₂) | 3.30 | kg |
| Dichloromethane (CH₂Cl₂) | 23 | L |
| 3,4-Dihydro-2H-pyran (C₅H₈O) | 7.57 | kg |
| Pyridinium 4-toluenesulfonate (C₁₂H₁₃NO₃S) | 45.2 | g |
| Developing agents: | Petroleum ether:Ethyl | |
| 1) Petroleum ether | acetate = 2:1 | |
| (mainly composed of pentane and hexane) | | |
| 2) Ethyl acetate (C₄H₈O₂) | | |
| Sodium hydroxide (NaOH) Solution | 30.0 | L |
| Water (H₂O) | 30.0 | L |
| Saturated NaCl Solution | 15.0 | L |
| Anhydrous Magnesium sulfate (MgSO₄) | 1.0 | kg |
| Methanol (CH₃OH) | 17 | L |

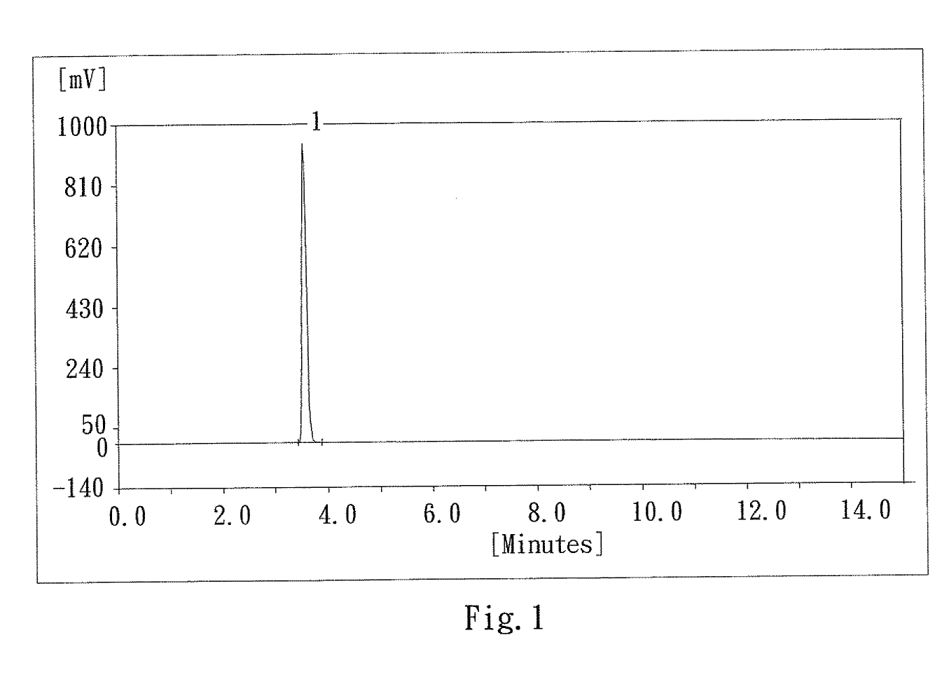

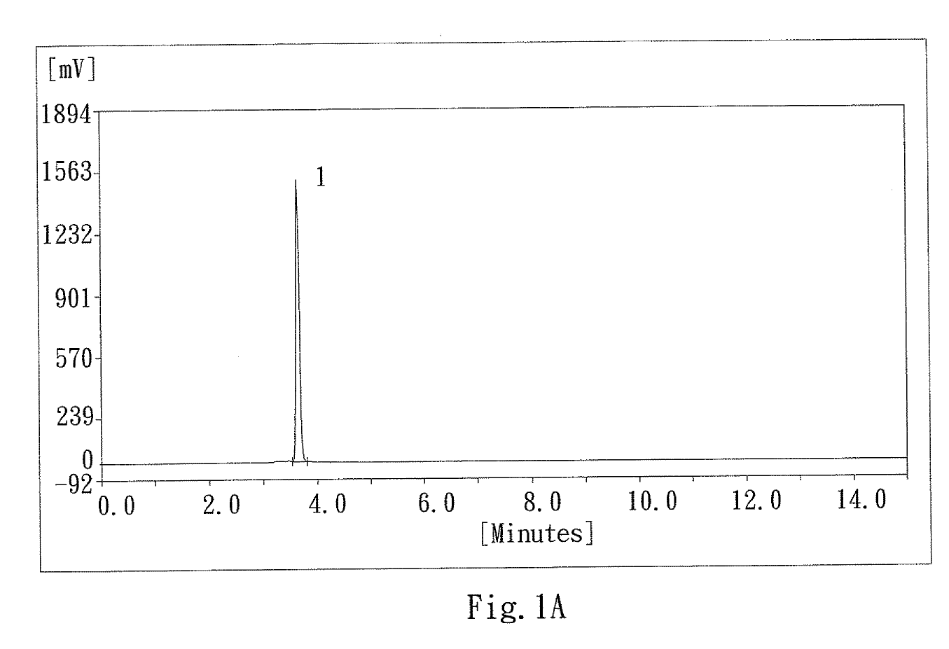

The product obtained after Step 4 of the first embodiment was analyzed by high performance liquid chromatography (hereinafter referred to as the HPLC), with UV1201 UV/Visible Wavelength Detector (220V), provided by Dalian Elite Analytical Instruments Co., Ltd., China. The detected results are shown in FIG. 1. The detector was operated with the run time of 15 minutes, Lambda of 288 nm and the column temperature of 25° C. The method information includes the reversed phase column modeled C18 (Discovery C 18, length of 250 mm, and diameter of 4.6 mm) and the flow of 1.0 ml/minute. The mobile phase (Buffer B) was evaluated by chromatographic grade acetonitrile (CH₃CN). The evaluation was begun from 100% acetonitrile that was reduced with a constant gradient over 15 minutes. Therein, Substance 1 of FIG. 1 is the prodrug composition for skin as described in the present invention (i.e. Deoxy-Arbutin Plus, DAP). The data are given in Table 2.

TABLE 2
Substance 1 of First Embodiment Obtained by HPLC

| | Retention Time | Peak Height | Peak Area | Area Percentage |
|---|---|---|---|---|
| Substance | (min) | (mV) | (mV · sec) | (%) |
| 1 | 3.57333 | 943.99 | 5875.38 | 100.0000 |

In Table 2, “Retention Time” refers to a time period from where the isolated sample component was introduced to where the maximum concentration of the component occurred, or from where the sample was introduced to where the apex of the chromatographic peak of a certain component appeared. Such a time period is the retention time of the component and represented by tR in the unit of minutes (min). “Peak Height” refers to the distance between the chromatographic peak apex and the chromatographic peak base signal value, wherein the chromatographic peak apex is the signal value output by the detector when the component to be detected is eluted from the column to its maximum concentration, and is typically expressed by nun or the signal unit of detectors. The peak height is a useful basis for quantitative determination. “Peak Area” refers to the area defined by the peak and the peak base. “Area Percentage” refers to a percentage of an area of a certain peak with respect to the total area of all the peaks.

### Second Embodiment

A second embodiment of the present invention is meta-deoxy-arbutin plus of formula (II), with a synthetic route shown below,

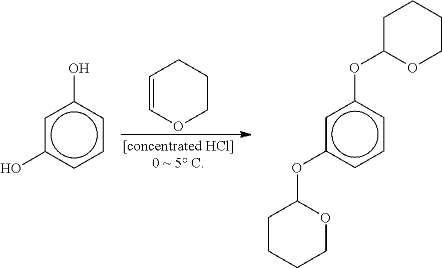

Along the synthetic route, Benzene-1,3-diol and 3,4-Dihydro-2H-Pyran were used as main materials, and concentrated HCl was sued as the catalyst, while liquid diheptyl phthalate (DHP) was used as the reactive solvent. The synthetic scale of the second embodiment was of 30 mole with conditions of 12 hours and 0˜5° C. The product of the above synthesis was then washed with 10% sodium hydroxide solution before crystallized and purified by ethoxyethane.

### Step 1 of Second Embodiment

13.8 g of solid Benzene-1,3-diol (1.0 eq) was weighted and dispersed in 42.1 g (4.0 eq) of liquid diheptyl phthalate (DHP). Then at 0˜5° C., liquid 3,4-Dihydro-2H-Pyran was added at an amount of 7.57 kg and 2 drops of concentrated HCl was also added. Afterward, the mixture was stirred for 2 hours before naturally coming to the room temperature. The mixture was the further stirred for 10 hours.

### Step 2 of Second Embodiment

The reaction was stopped and 50 ml of ethoxyethane was added and dissolved by stirring. The organic phase was washed with 10% sodium hydroxide solution for six times (each time with 50 ml of 10% sodium hydroxide solution) and then washed with water for three times (each time with 50 ml of water) to make it into neutral aqueous phase. Afterward, the organic phase was washed with saturated NaCl solution for three times (each time with 50 ml of saturated NaCl solution). Anhydrous magnesium sulfate was added to dry the organic phase.

### Step 3 of Second Embodiment

After magnesium sulfate was removed by filtering, the organic phase was concentrated under reduced pressure so as to remove ethoxyethane and excessive 3,4-Dihydro-2H-Pyran.

### Step 4 of Second Embodiment

Finally, 29.7g meta-Deoxy-Arbutin Plus was obtained with a yield of 85%.

TABLE 3
List of Components of Second Embodiment

| Component | | |
|---|---|---|
| (Chemical Formula/IUPAC Name) | Amount | |
| Benzene-1,3-diol | 13.8 | g |
| (C₆H₄(OH)₂/Benzene-1,3-diol) | | |
| Diheptyl phthalate | 42.1 | g |
| 3,4-Dihydro-2H-pyran (C₅H₈O) | 7.57 | kg |
| Concentrated Hydrochloric acid (HCl) | 2 | drops |
| Ethoxyethane ((C₂H₅)₂O) | 50 | ml |
| Sodium hydroxide (NaOH) Solution | 300 | ml |
| Water (H₂O) | 150 | ml |
| Saturated NaCl solution | 150 | ml |
| Anhydrous magnesium sulfate (MgSO₄) | 1.0 | kg |

The product obtained after Step 4 of the second embodiment was analyzed by the same high performance liquid chromatography (hereinafter referred to as the HPLC). The detected results are shown in FIG. 1A. The detector was operated with the parameters similar to those used for the first embodiment except that Lambda was changed to 274 nm. The method information was identical to that of the first embodiment. Substance 1 of FIG. 1A is the prodrug composition for skin as described in the present invention (i.e. Deoxy-Arbutin Plus, DAP). The data are given in Table 4.

TABLE 4
Substance 1 of Second Embodiment Obtained by HPLC

| | Retention Time | Peak Height | Peak Area | Area Percentage |
|---|---|---|---|---|
| Substance | (min) | (mV) | (mV · sec) | (%) |
| 1 | 3.62667 | 1512.91 | 6705.62 | 100.0000 |

For the prodrug composition for skin as described in the present invention, quantitative structure-activity relationship (QSAR) was adopted to determine the lipid/water partition coefficient of the compound, as shown in the following Table 5. In this table, Log P is the pharmacokinetic parameter related to drug permeability, and is the partition coefficient of the organic compound in octanol and in water. A value that is smaller than 5 suggests desired drug permeability. The greater the Log P is, the more lipophilic and the less hydrophilic the substance is to the organic phase. In the present invention, hydroquinone, arbutin, deoxy-arbutin and the disclosed DAP were compared, and it is the finding that the disclosed DAP exhibited the highest lipid/water partition coefficient. That is, the disclosed DAP is most lipophilic to the organic phase among the tested substances, thus being better water resistant.

TABLE 5
Hydroquinone, Arbutin, Deoxy-Arbutin and
DAP's Lipid/Water Partition Coefficients

| Compounds | Calc. LogP | Calc. CLogP |
|---|---|---|
| Hydroquinone (HQ) | 0.56 | 0.808 |
| Arbutin | −0.58 | −0.508 |
| Deoxy-Arbutin | 2.36 | 1.96 |
| Instant Formula (I): Deoxy-Arbutin Plus | 3.47 | 2.92 |
| (DAP) | | |

In application, the prodrug composition of the present invention may have the composition of formula (I) incorporated into a preparation, so as to put the prodrug composition into cosmetic products or medical products to be applied to the skin, thereby delivering the disclosed DAP. The preparation may be in the form of cream, emulsion, composite emulsion, anhydrous composition, aqueous dispersion, grease, ointment, balm, liniment, gel, gel cream, aqueous alcoholic solution, shampoo, hair conditioner or mousse.

Therein, the suitable cosmetic products may include eye concealer, powder, makeup remover, cream and eye shadow. An example cosmetic product according to the present invention may have the formula given in Table 6.

TABLE 6
Formula of Cosmetic Product used in Present Invention

| | | Wt Percentage |
|---|---|---|
| Component | INCI NAME | (%) |
| Mineral oil | Paraffinum Liquidum | 6 |
| TN | C12-15 Alkyl Benzoate | 4 |
| Avacado oil | Persea Gratissima (Avocado) Oil | 3 |
| Emulsifier & | C13-14 isoparaffin, Laureth-7, | 3 |
| Thickener | Polyacrylamide | |
| (305)/(for cream) | | |
| Shea Butter | Butyrospermum Parkii (Shea | 2 |
| | Butter) Fruit | |
| Disclosed | Deoxy-Arbutin PLUS | 2 |
| Deoxy-Arbutin Plus | | |
| (DAP) | | |
| Butylene Glycol (1, | Butylene Glycol | 2 |
| 3BG) | | |
| Rose Water | Cabbage Rose Distilled | 2 |
| Red alga Extract | Corallina Officinalis Extract | 2 |
| Liquorice Juice | Liquorice Distilled | 2 |
| Loofah Extract | Luffa Cylindrica extract | 2 |
| 16 Alcohol | Cetyl Alcohol | 0.8 |
| 18 Alcohol | Stearyl Alcohol | 0.8 |
| Preservative | Phenoxyethanol | 0.2 |
| HA | Sodium hyaluronate | 0.05 |
| Softened Water | Aqua | 68.15 |

In the formula of Table 6, the soften water takes a relatively large share and its use amount may be adjusted in view of the other components to make the sum become 100%. The table is provided as a preferred embodiment without limiting intention. In other embodiments of the present invention, the amount of DAP may range between 0.001% and 20%, according to practical needs. No change in color of the preparation of the proposed formula was observed after six-month storage at the room temperature. SGS (SGS Group—Societe Generale de Surveillance) test results also demonstrated that there was no release of HQ. Thus, it is proven that the formula of Table 6 with the existence of the disclosed DAP is more stable than formulas containing HQ and/or DA.

Moreover, for proving the whitening effect of the DAP-containing prescription according to the present invention, a clinical test was performed and used the detection device shown in Table 7. Six subjects joining the test received the cream preparation containing 1% or 2% DAP on facial freckles and the normal facial skin twice a day for consecutive four weeks (2010 Nov. 24-2010 Dec. 15), and the whitening effect was evaluated.

TABLE 7
Specifications of Adopted Skin Analysis System
SMART SKIN ANALYSIS SYSTEM

Model: SMART U-130
Origin: Taiwan
Dimensions: L120 × W55 × H30 (mm)
Input Voltage: 110 V/60 Hz
Accessory: 50x lens (Diameter: 3.8 cm, Length: 4.7 cm)
Illumination: Normal light and polarized light (for experiments) Available
to use x50 optical lens.

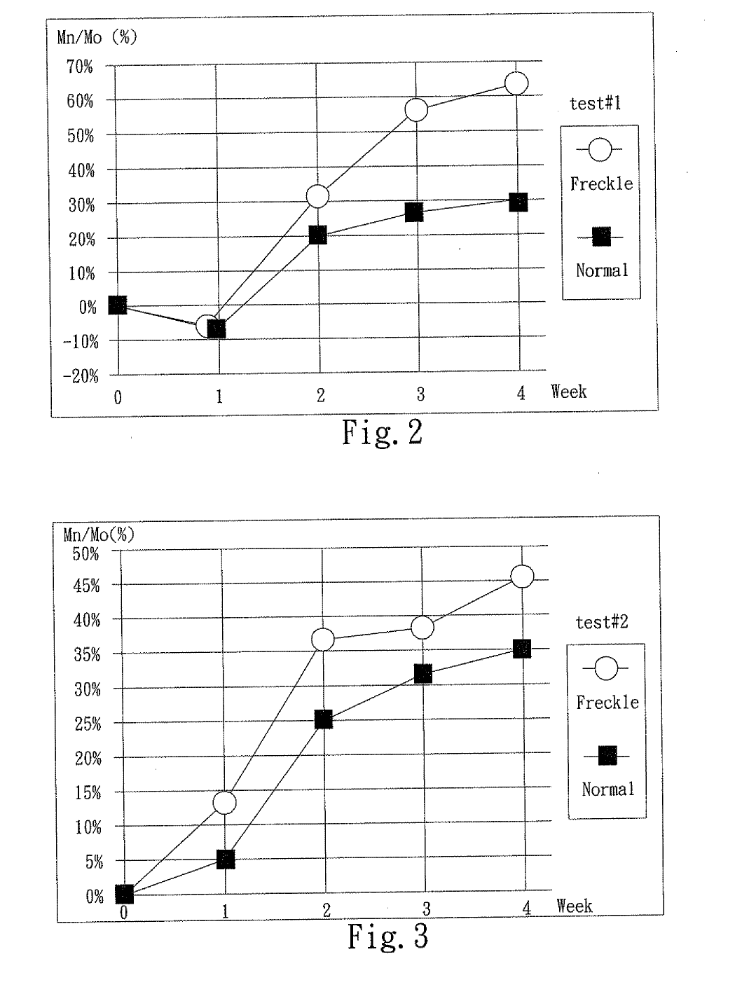

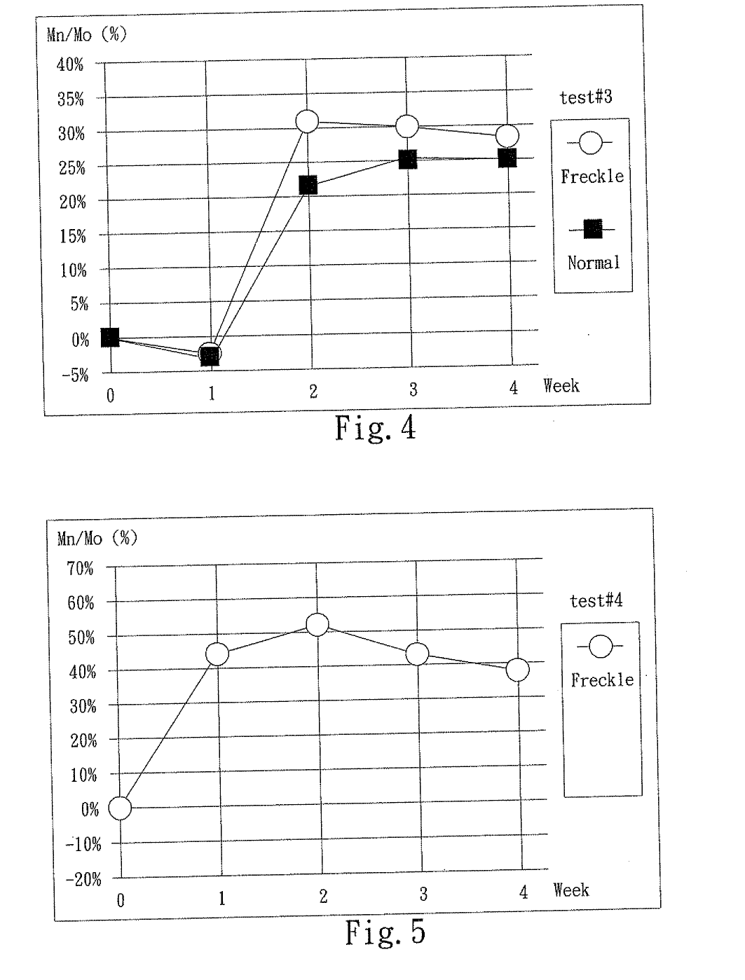

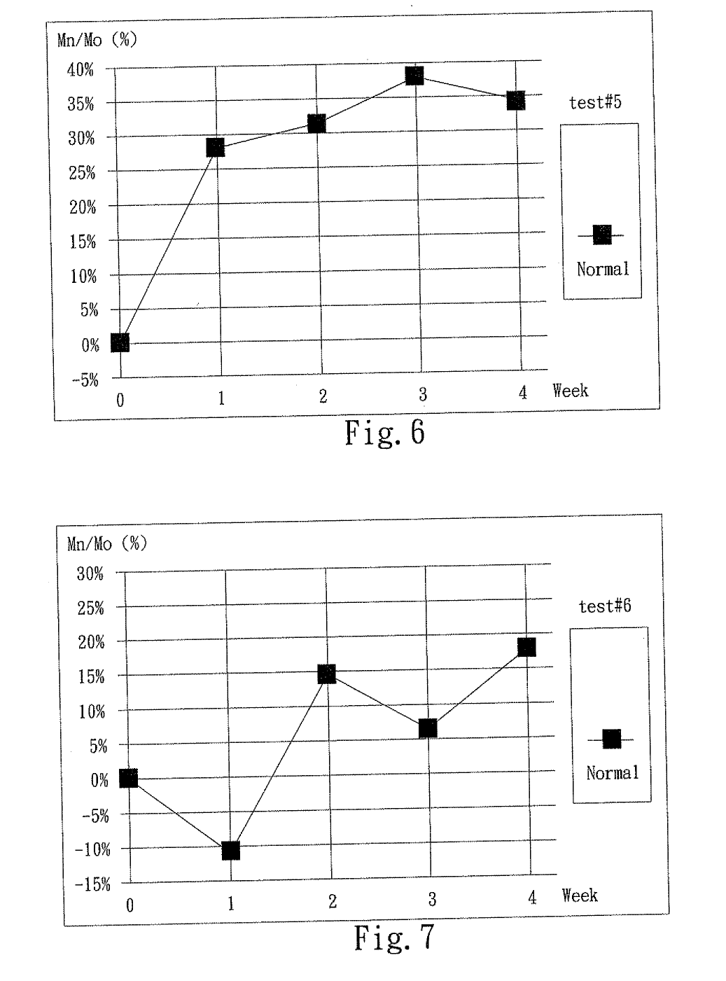

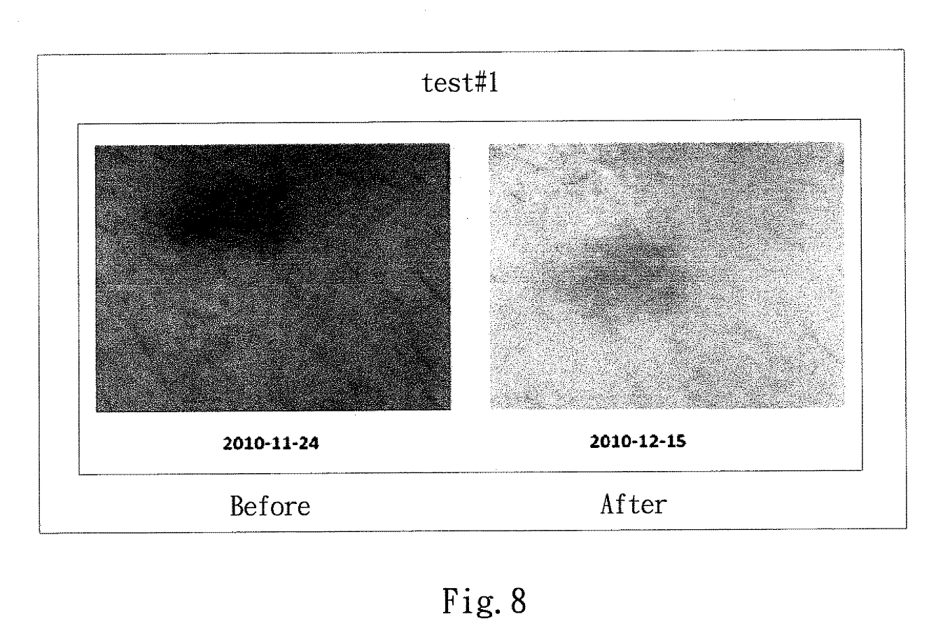

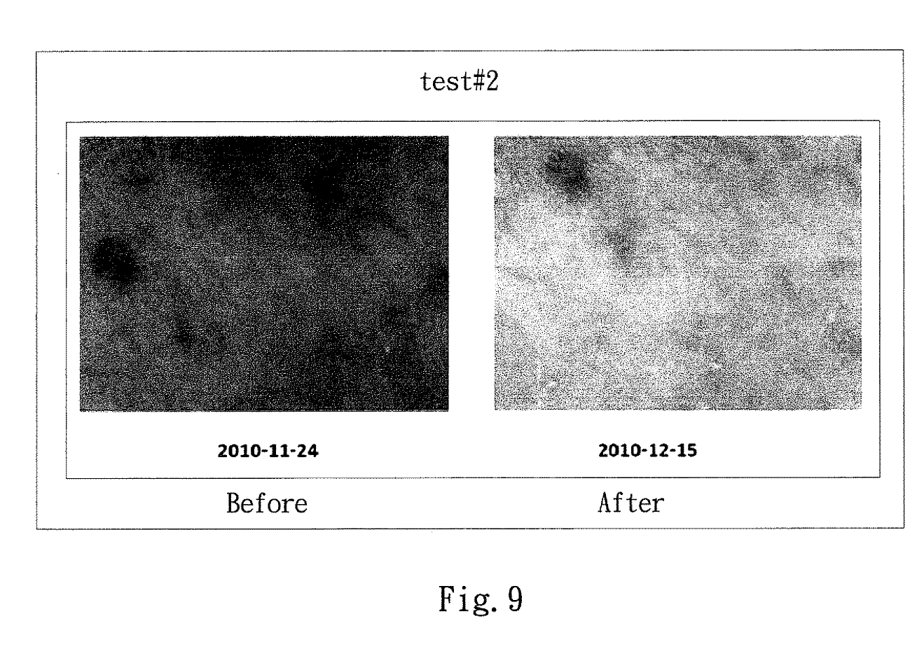

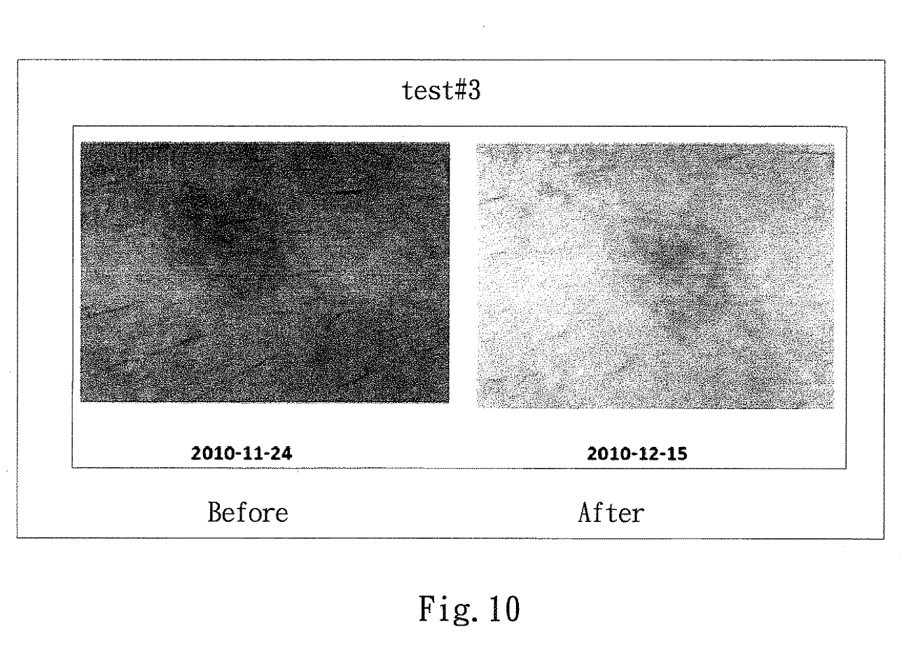

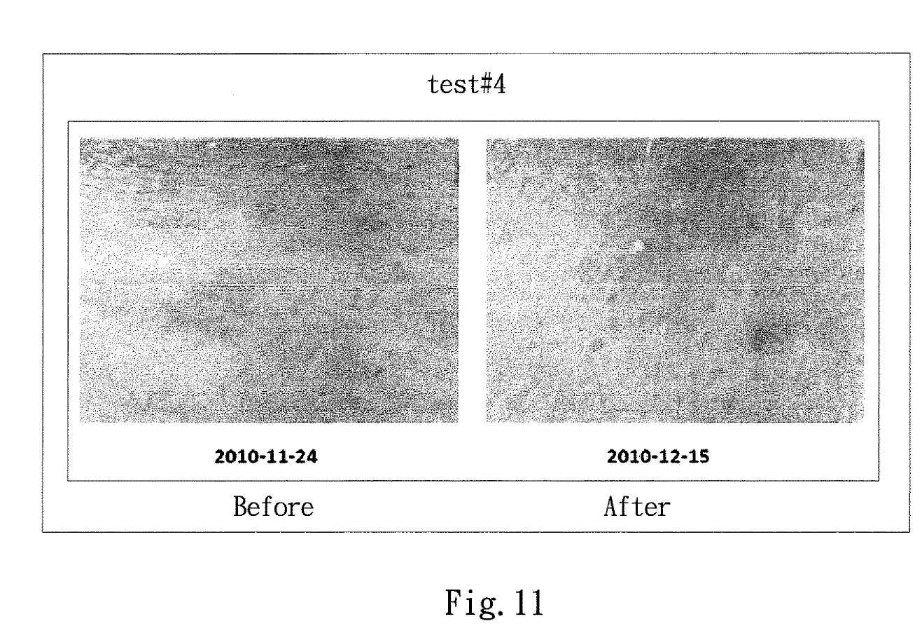

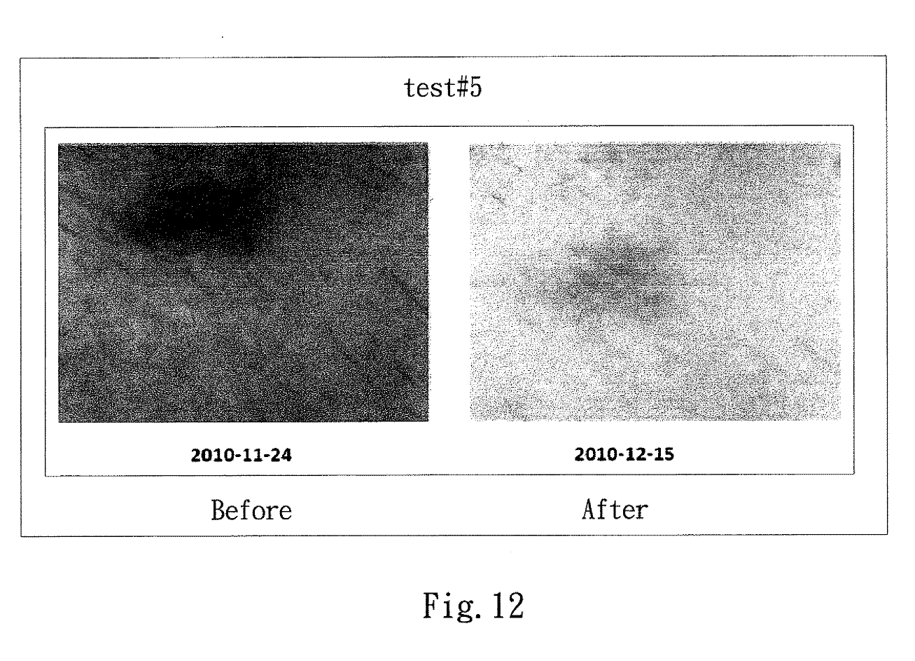

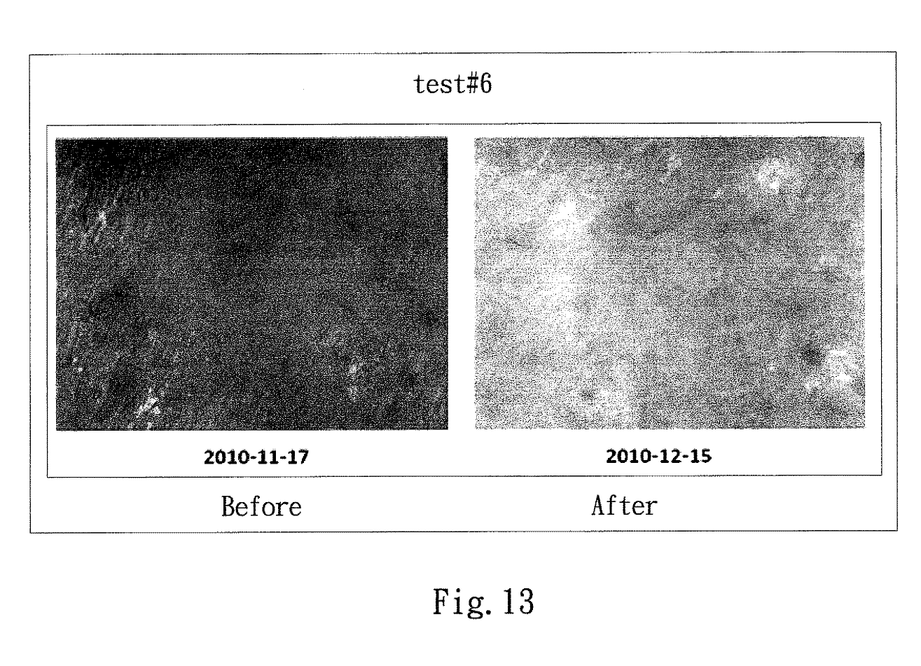

According to the foregoing clinical test, data graphs with respect to the disclosed prodrug composition as shown in FIG. 2 through FIG. 7 have been made, while the photographs showing the tested sites of several subjects are reflected in FIG. 8 through FIG. 13. The subjects for the tests are described below:

[1] Test#1: referring to FIG. 2 and FIG. 8, “freckles in the left cheek” and the normal facial skin were subjects on which a prescription containing 2% of the disclosed DAP was applied for consecutive four weeks. The prescription exhibited a whitening effect on the freckles up to 63%, and a whitening effect on the normal skin up to 29%.

[2] Test#2: referring to FIG. 3 and FIG. 9, “solar lentigines in the left cheek” and the normal facial skin were subjects on which a prescription containing 2% of the disclosed DAP was applied for consecutive four weeks. The prescription exhibited a whitening effect on the solar lentigines up to 46%, and a whitening effect on the normal skin up to 35%.

[3] Test#3: referring to FIG. 4 and FIG. 10, “freckles in the left cheek” and the normal facial skin were subjects on which a prescription containing 2% of the disclosed DAP was applied for consecutive four weeks. The prescription exhibited a whitening effect on the freckles up to 28%, and a whitening effect on the normal skin up to 25%.

[4] Test#4: referring to FIG. 5 and FIG. 11, “freckles in the right cheek” were subjects on which a prescription containing 2% of the disclosed DAP was applied for consecutive four weeks. The prescription exhibited a whitening effect on the freckles up to about 39%.

[5] Test#5: referring to FIG. 6 and FIG. 12, “the normal skin in the left cheek” was the subject on which a prescription containing 2% of the disclosed DAP was applied for consecutive four weeks. The prescription exhibited a whitening effect on the normal skin up to about 34%.

[6] Test#6: referring to FIG. 7 and FIG. 13, “the normal skin in the left cheek was the subject on which a prescription containing 1% of the disclosed DAP was applied for consecutive four weeks. The prescription exhibited a whitening effect on the normal skin up to 19%.

The four-week clinical test as mentioned above demonstrates that the disclosed DAP is indeed effective in improving facial freckles, suppressing the activity of tyrosinase in the skin and inhibiting melanin synthesis, by directly combining with tyrosinase to accelerate melanin's decomposition and removal, so as to reduce skin pigmentation without side effects such as being toxic to melanocytes and irritative as well as allergenic to the skin.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A prodrug composition for skin with twin protecting groups bound by disubstituted benzene, the prodrug composition having a composition representing by the following formula (I), 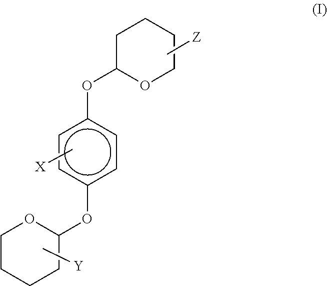
where,
X is an independent benzene ring compound selected from disubstituted benzene or benzoic acid derivatives;
Y is an independent heterocyclic compound with three- to six-membered rings, and is selected from the group consisting of an oxygen containing heterocyclic compound and a sulphur containing heterocyclic compound; and
Z is an independent heterocyclic compound with three- to six-membered rings, and is selected from the group consisting of an oxygen containing heterocyclic compound and a sulphur containing heterocyclic compound, while the Z and the Y are identical or different heterocyclic compounds.

2. The prodrug composition of claim 1, wherein the prodrug composition comprises one of a para-phenylene structure, a meta-phenylene structure and an ortho-phenylene structure.

3. The prodrug composition of claim 1, wherein the Y is tetrahydropyran.

4. The prodrug composition of claim 1, wherein the Z is tetrahydropyran.

5. The prodrug composition of claim 1, wherein the Y has an independent chain compound substituting for the heterocyclic compound, and the chain compound is selected from the group consisting of acetyl, methyl, methoxy and methyl ether derivatives.

6. The prodrug composition of claim 1, wherein the Z has an independent chain compound substituting for the heterocyclic compound, and the chain compound is selected from the group consisting of acetyl, methyl, methoxy and methyl ether derivatives.

7. The prodrug composition of claim 1, wherein the prodrug composition is present as a preparation such that the prodrug composition for skin is allowed to be incorporated in a cosmetic product for application, and the preparation has a form selected from the group consisting of cream, emulsion, composite emulsion, anhydrous composition, aqueous dispersion, grease, ointment, balm, liniment, gel, gel cream, aqueous alcoholic solution, shampoo, hair conditioner and mousse.

8. The prodrug composition of claim 7, wherein the cosmetic product is selected from the group consisting of eye concealer, powder, makeup remover, cream and eye shadow.
